(19) United States
(12) Patent Application Publication
(10) Pub. No.: US 2007/0259007 A1
(43) Pub. Date: Nov. 8, 2007

(54) Lactoferrin: an adjuvant for vaccines

(76) Inventors: Marian L. Kruzel, Houston, TX (US); Jeffrey K. Actor, Houston, TX (US)

Correspondence Address: Kurt S. Myers, 7634 Braesdale Ln., Houston, TX, 77071, US

(21) Appl. No.: 11/810,477

(22) Filed: Jun. 6, 2007

Related U.S. Application Data
(63) Continuation-in-part of application No. PCT/US05/44871, filed on Dec. 9, 2005.
(63) Continuation-in-part of application No. 10/023,096, filed on Dec. 18, 2001.
Application No. 10/023,096 is a division of application No. 09/932,190, filed on Aug. 17, 2001, now Pat. No. 6,455,687.
Application No. 09/932,190 is a division of application No. 09/421,632, filed on Oct. 19, 1999, now Pat. No. 6,277,817.
(60) Provisional application No. 60/635,414, filed on Dec. 10, 2004.

(51) Int. Cl. A61K 45/00; A61P 43/00

(52) U.S. Cl. 424/278.1

(57) ABSTRACT

The method of the present invention provides a novel use of Lactoferrin as an adjuvant composition suitable to be used in vaccines. More specifically, the present invention is directed to the use of Lactoferrin to augment a vaccine efficacy by generation of T lymphocytes and B lymphocytes response and subsequent protection against challenge with a virulent pathogen, cancer or allergic antigens (allergens). Also provided by the present invention is a method of manufacture of the adjuvant and it use as prophylactic or therapeutic agents.

### CROSS-REFERENCE TO RELATED APPLICATIONS

This application is a continuation-in-part of co-pending International Application No. PCT/US2005/044871 filed Dec. 9, 2005, which designates the U.S. and claims the benefit of U.S. Provisional Patent Application No. 60/635,414 filed Dec. 10, 2004. This application is also a continuation-in-part of co-pending U.S. patent application Ser. No. 10/023,096 filed Dec. 18, 2001, which is a divisional of U.S. patent application Ser. No. 09/932,190 filed Aug. 17, 2001 (now U.S. Pat. No. 6,455,687). U.S. Pat. No. 6,455,687 is a divisional of U.S. patent application Ser. No. 09/421,632 filed Oct. 19, 1999 (now U.S. Pat. No. 6,277,817). The disclosures of the aforesaid applications and patents are hereby incorporated herein by reference.

### STATEMENT REGARDING FEDERALLY SPONSORED RESEARCH OR DEVELOPMENT

The U.S. Government has a paid-up license in this invention and the right in limited circumstances to require the patent owner to license others on reasonable terms as provided for by the terms of Grant No. 1 R41 AI51050-01A1 and 2 R42 AI051050-02, awarded by the National Institutes of Health (National Institute of Allergy and Infectious Diseases).

### FIELD OF INVENTION

The present invention relates to lactoferrin as an adjuvant composition suitable to be used in vaccines or in conjunction with administered vaccines. In particular, lactoferrin is used to augment a vaccine efficacy and subsequent protection against challenge with a virulent pathogen, or encounter with a toxin entity. A specific aspect of the invention is that the human recombinant lactoferrin of the present invention is being glycosylated such as to be the same as endogenous lactoferrin released in the body, hence making lactoferrin suitable for injection with a vaccine.

### BACKGROUND OF THE INVENTION

A vaccine is an antigenic preparation used to establish specific immunity to a disease. The immune system recognizes vaccine agents as foreign, destroys them, and ‘remembers’ them. When the virulent version of an agent comes along, the immune system is thus prepared to respond, by a) neutralizing the target agent before it can enter cells, and b) by recognizing and destroying infected cells before that agent can multiply to vast numbers. Vaccines have contributed to the eradication of smallpox, one of the most contagious and deadly diseases known to man. Other diseases such as rubella, polio, measles, mumps, chickenpox, and typhoid are nowhere near as common as they were just a hundred years ago.

Vaccines can be prophylactic (e.g. to prevent or ameliorate the effects of a future infection by any naturally occurring pathogen; to reduce toxic effects of released bacterial toxins; to ameliorate or prevent autoimmune disorders; to limit allergic responses; to prevent or limit cancerous events), or therapeutic (e.g. vaccines against cancer).

There are four major classes of traditional vaccines: a) vaccines containing killed microorganisms—examples are vaccines against flu, cholera, bubonic plague, and hepatitis A; b) vaccines containing live, attenuated microorganisms—examples include yellow fever, measles, rubella, Bacille Calmette-Guérin (BCG) and mumps; c) toxoids—these are inactivated toxic compounds from micro-organisms in cases where these cause illness—examples of toxoid-based vaccines include tetanus and diphtheria; d) subunit—rather than introducing a whole inactivated or attenuated micro-organism to an immune system, a fragment of it can create an immune response—examples include the subunit vaccine against hepatitis B virus (HBV) that is composed of only the surface proteins of the virus (produced in yeast) and the virus like particle (VLP) vaccine against Human Papillomavirus (HPV) that is composed of the viral major capsid protein.

Recently a novel approach has been taken to develop unconventional vaccines, such as: a) conjugate—example Haemophilus influenzae type B vaccine; b) recombinant vector—by combining the physiology of one micro-organism and the DNA of the other, immunity can be created against diseases that have complex infection processes; c) DNA vaccination—it works by insertion (and expression, triggering immune system recognition) into human or animal cells, of viral or bacterial DNA. Some cells of the immune system that recognize the proteins expressed will mount an attack against these proteins and cells expressing them.

Also, a new concept on vaccines against cancer, autoimmune disorders and allergic responses has been actively explored with the aim of prophylactic and/or therapeutic uses. The cancer therapeutic vaccines, are designed to treat cancer by stimulating the immune system to recognize and attack specific cancer cells, taking the advantage that certain molecules on the surface of cancer cells are either unique or more abundant than those found on normal or non-cancerous cells. When a vaccine containing cancer-specific antigens is injected into a patient, these antigens will stimulate the immune system to attack cancer cells, reducing growth and spread without harming normal cells. The cancer prophylactic vaccines are given to individuals at risk to develop specific type of cancer to stimulate the immune system which can (1) attack cancer-causing viruses and prevent viral infection, and (2) induce cellular responses to limit uncontrolled growth of cancerous cells overexpressing specific cell surface markers (antigens). Currently, two vaccines have been approved by FDA to prevent virus infections that can lead to cancer: the hepatitis B vaccine, an infectious agent associated with liver cancer; and Gardasil™, which prevents infection with human papilliomavirus (HPV) associated with the cervical cancer.

Immunization can assist in development of control regulated towards specific allergic responses. There is a growing evidence to support the role of regulatory T cells in controlling the development of allergic diseases. As an example, a novel anti-allergy vaccine has been developed (e.g. against ragweed pollen). These vaccines cause the body to produce allergen-attacking antibodies. Similarly, a tolerogenic vaccine has been proposed for autoimmune disorders, using antigen-specific immature dendritic cells. An example would be that used to fight against development of skin disorders, such as with atopic dermatitis.

Immunization can also assist in protection against development of autoimmune disorders. Similarly, immunization can protect against progression of existing autoimmune disease (therapeutic vaccines). Examples include experimental vaccines being developed against organ specific and sytemic autoimmune disorders. These vaccines would limit the pathogenesis of autoimmune diseases, such as myasthenia gravis.

An ideal vaccine should contain components acting to direct immune response of the host in a manner similar to that seen in a natural infection. The vaccine should induce and direct specific response by T lymphocytes for delayed type hypersensitivity function and for cytotoxic function, and by B lymphocytes for antibody production. Indeed, in the absence of adjuvants, modified, non-pathogenic forms of an infectious agent are able to stimulate the immune system in a similar manner to that seen with the wild type or pathogenic agent. Historically, some of the most protective vaccines, such as the polio vaccine and BCG, differ only slightly from their parental disease-causing organism and yet portray a lack of pathogenicity. Unfortunately, it is not always possible to develop such attenuated vaccines for all viral, bacterial or parasitic pathogens. An alternative is to use killed organisms as vaccines, but these, while useful in the short term, often fail to induce long-lasting cell mediated immunity (CMI). Indeed, there is a greater chance that humoral immunity (antibody-based) will develop. This is also the major occurrence when using highly purified antigens or subunit vaccines. Therefore, adjuvants are typically used to increase immunogenicity and T lymphocyte response to antigens.

Three classes of adjuvants have long been used to augment immune responses in vaccination (Allison AC. Immunological adjuvants and their modes of action. Arch Immunol Ther Exp 1997; 45:141-147). These can be classified as (i) vegetal, like saponin or glucan extract; (ii) bacterial like monophosphoryl lipid A, trehalose dimycolate, cholera toxin or lipopolysaccharides and their derivatives; or (iii) chemical like aluminium hydroxide, surfactants, emulsions, or micro and nanoparticles. Currently the only adjuvants approved for human use are the aluminum salts. Alum has been effective for some vaccines, but in many instances it has limited capacity for responses other than those which are humoral mediated. The prototype adjuvant for cell mediated and DTH responses is complete Freund's adjuvant (CFA), consisting of a water-in-oil emulsion containing approximately 50% mineral oil, emulsifying agent Arlacel A, and killed mycobacterium.

Although CFA is an effective adjuvant for generation of cell mediated response, it is undesirable in animals and unacceptable for human use due to its severe side effects, including pain, abscess formation, local necrosis, and fever. Omission of the Mycobacteria generates incomplete Freund's adjuvant (IFA), which reduces both the immune response and the side effects. Even still, side effects associated with IFA, predominantly formation of granuloma at the vaccination site, are still considered too severe for general human use. Thus, the goal of adjuvant research in the last 40 years has been to find effective, nontoxic substitute for both the vehicle (mineral oil and Arlacel A) and the immunostimulatory components (mycobacterial cell wall components) of CFA while maintaining adjuvant activity. Examples of alternative vehicles are liposomes, immune-stimulating complexes and microfluidized squalene-in-water emulsions (Powell, M F, and Newman, M J. Vaccine Design:subunit & Adjuvant Approach. Plenum Pub Corp, 1995.). More recently, adjuvant formulations have been developed that contain molecules to directly augment cytokine production. Examples are a synthetic muramyl dipeptide analog or monophosphoryl lipid A. In addition, novel adjuvants have been developed which may be customized to augment appropriate immune responses. For instance, research from our laboratory on poloxamers suggest that copolymers with 10% polyoxyethylene (POE) preferentially augment Type 2 helper T-lymphocyte responses which support antibody responses, while copolymers with <10% POE augment both Type 1 and Type 2 helper T-lymphocyte responses supporting a broader range of antibody and cellular immune responses (Newman M J, Actor J K, Balusubramanian M, Jagannath C. Use of nonionic block copolymers in vaccines and therapeutics. Crit Rev Ther Drug Carrier Syst. 1998; 15(2):89-142; Todd C W, Balusubramanian M, Newman M J. Development of adjuvant-active non-ionic block copolymers. Adv Drug Deliv Rev. 1998; 6;32(3):199-223).

The requirement for safety and efficacy in adjuvants, with an acceptable balance of risk versus benefit, is of paramount importance and determines the framework within which adjuvant development must occur. Ideally, the adjuvant is a biodegradable natural substance which causes highly specific immune responses by activating only the specific population of cells in lymphoid tissue that are needed for the desired immune effect. As presented in the parent application, lactoferrin may meet the criteria of the ideal adjuvant for general use in the vaccination protocols.

Therefore, the present invention is illustrated by the use of lactoferrin as an adjuvant with tuberculosis (TB) vaccine.

Tuberculosis (TB) is the leading cause of morbidity due to an infectious disease and is a serious, unresolved burden upon the world's population despite aggressive vaccine implementation and progressive antibiotic treatment. The causative agent is Mycobacterium tuberculosis (MTB), an intracellular bacterium whose primary host cell is the macrophage. It is estimated that over a third of the world's population is infected with MTB, with incidence of infection continuously on the rise (World-Health-Organization. 2007. Global Tuberculosis Control. Surveillance, Planning, Financing. The World Health Report 2007. World Health Organization, Geneva, Switzerland, ISBN-13 9789241563390). The 2007 World Health Organization (WHO) report on tuberculosis (TB) incidence rates around the world shows dense epidemic areas where TB vaccine is widely applied clearly illustrating the limitations of the BCG vaccine towards spread of disease (Lietman, T., and S. M. Blower. 2000. Potential impact of tuberculosis vaccines as epidemic control agents. Clin Infect Dis 30 Suppl 3:S316). Declining BCG efficacy will continue to be an obstacle in eradicating tuberculosis; a modeling study predicted that a MTB vaccine with only 50% efficacy would save thousands of lives in the next 10 years (Murray, C. J., and J. A. Salomon. 1998. Modeling the impact of global tuberculosis control strategies. Proc Natl Acad Sci USA 95:13881). Accompanying strategies for the eradication of TB have included aggressive managed treatment, such as the directly observed treatment short course program (DOTS), however, dramatic strides in improving TB incidences will only occur with the development of improved vaccines and adjuvants.

Protection against MTB infection requires host generation of a strong cell mediated immunity (CMI); specifically, a T-cell mediated delayed type hypersensitivity (DTH) response, involving the activation of both CD4+ and CD8+ lymphocytes and development of a strong T-cell helper type-1 (TH1) response as indicated by production of interferon gamma (IFN-γ) and macrophage-, dendritic cell-, or NK cell-derived interleukin-12 (IL-12), activating host macrophages and leading to bacterial clearance (Flynn, J. L. 2004. Immunology of tuberculosis and implications in vaccine development. Tuberculosis (Edinb) 84:93 and Bloom, B. R., J. Flynn, K. McDonough, Y. Kress, and J. Chan. 1994. Experimental approaches to mechanisms of protection and pathogenesis in M. tuberculosis infection. Immunobiology, 191:526). DTH contributes to the development of protective granulomatous response, limiting organism dissemination. The widely used TB vaccine is a live attenuated strain of Mycobacterium bovis Bacillus Calmette-Guerin (BCG). The global efficacy of BCG in generating a protective host response against MTB has fallen, especially in regards to preventing adult onset disease. BCG as a live vaccine has historically been more efficacious than killed or subunit vaccines by being able to induce CMI required for protection. BCG remains the gold standard by which other tuberculosis vaccines are judged. Advantages of the BCG vaccine lie in its potential ability to persist in vivo for longer periods of time and to induce prolonged immunological memory, thus generating CMI and immune responses at mucosal surfaces at relatively low cost. However, efficacy of BCG has fallen during the past two decades, especially in protection against adult pulmonary tuberculosis. Clinical studies indicate a range of efficacy from 80% effective in the United Kingdom to almost 0% efficacy in India, Japan, and Malawi (Brennan, M. 2004. A new generation of tuberculosis vaccine. In Vaccines: Preventing Disease & Preventing Health. C. A. de Quadros, ed. Pan American Health Organization, Washington, D.C., p. 177 and Colditz, G. A., T. F. Brewer, C. S. Berkey, M. E. Wilson, E. Burdick, H. V. Fineberg, and F. Mosteller. 1994. Efficacy of BCG vaccine in the prevention of tuberculosis. Meta-analysis of the published literature. Jama 271:698). The reasons for this are unclear at this time. The most important problem with the current BCG vaccine is its inability to generate and sustain the necessary protective DTH and CMI responses needed for control of MTB infection. It is also clear that there is a great need to develop novel vaccines or adjuvants that can overcome the failure of BCG, offering protection against infection and limiting dissemination of organisms.

To date, no research vaccine against tuberculosis has been able to consistently surpass BCG in reduction of lung organisms following challenge with virulent MTB (McMurray, D. N. 2003. Recent progress in the development and testing of vaccines against human tuberculosis. Int J Parasitol 33:547). Multiple alternative vaccines failed to protect against MTB. Indeed, surpassing BCG seemed an unrealistic goal that may never be accomplished. The need for new MTB vaccines is paramount, as evident by the number of new vaccine formulations currently in the process of Phase I clinical testing. These include, but are not limited to, vaccines utilizing live attenuated MTB, recombinant BCG with MTB Ag85 antigens, inactived M. vaccae organisms, DNA vaccines utilizing Heat Shock protein 65 DNA, dendritic vaccines incorporating Ag85-ESAT6 proteins, and modified (auxotrophic) organisms (Wang, J., and Z. Xing. 2002. Tuberculosis vaccines: the past, present and future. Expert Rev Vaccines 1:341 and Kumar, H., D. Malhotra, S. Goswami, and R. N. Bamezai. 2003. How far have we reached in tuberculosis vaccine development? Crit Rev Microbiol 29:297).

Employing adjuvants as a strategy to improve BCG vaccine efficacy is a major world-wide research focus. Most adjunct adjuvants, while effective in enhancing humoral immunity, fail to increase T-cell responses considered protective during subsequent MTB challenge. Presently, the model adjuvant capable of promoting generation of CMI and DTH responses is complete Freund's adjuvant (CFA); a water-in-oil emulsion containing 50% mineral oil, emulsifying agent Arlacel A, and heat-killed avirulent MTB strain, H37Ra. CFA is highly toxic; it is not suitable for human use and becoming more undesirable for use in animals. Recent advances in adjuvant development are directed towards generating vaccines that approach the efficacy of CFA while possessing none of its toxic properties (Brennan, M. 2004. A new generation of tuberculosis vaccine. In Vaccines: Preventing Disease & Preventing Health. C. A. de Quadros, ed. Pan American Health Organization, Washington, D.C., p. 177).

### BRIEF DESCRIPTION OF DRAWINGS

FIG. 1 shows that Lactoferrin increases IL-12 production from stimulated murine macrophages. J774A.1 (top) or RAW 264.7 cells (bottom) were stimulated with LPS (200 ng/ml) and increasing concentrations of Lactoferrin (1 to 1000 μg/ml). Supernatants were assessed for IL-12 (closed bar) and IL-10 (open bar). Average values (pg/ml) with standard errors are shown. *, p<0.05 compared to LPS alone.

FIG. 2 shows Stimulation (Proliferation) Index of splenocytes from mice immunized with BCG. Splenocytes from mice immunized one time with BCG in IFA (BCG/IFA), BCG in IFA with Lactoferrin (100 μg/mouse) (BCG/IAF/LF), or BCG in CFA (BCG/CFA) were assessed for proliferative response to Heat-Killed BCG. Average Stimulation Index values with standard errors are shown. Responses are compared to non-immunized control mice (Control). *, p<0.05; **, p<0.05 as measured by two-tailed unpaired Student's t-test for indicated comparisons.

FIG. 3 shows that BCG Immunization with adjunct Lactoferrin adjuvant augments proinflammatory mediators from splenocytes. Splenocytes from mice immunized one time with BCG in IFA (BCG/IFA), BCG in IFA with Lactoferrin (100 μg/mouse) (BCG/IFA/LF), or BCG in CFA (BCG/CFA) were assessed for response to Heat-Killed BCG. Supernatants were assessed by ELISA for TNF-α, IL-1β, and IL-6. Average values (pg/ml) with standard errors are shown. Responses are compared to non-immunized control mice (Control). *, p<0.05 vs non-immunized; **, p<0.05 vs. BCG alone; ***, p<0.05 CFA vs. Lactoferrin group.

FIG. 4 shows that adjunct Lactoferrin adjuvant increases TH1 cytokine IFN-γ from splenocytes, as well as TNF-a and IL-6 production. Splenocytes from mice immunized one time with BCG in IFA (BCG/IFA), BCG in IFA with Lactoferrin (100 μg/mouse) (BCG/IFA/LF), or BCG in CFA (BCG/CFA) were assessed for production of IFN-γ, TNF-a, or IL-6 in response to Heat-Killed BCG. Average values (pg/ml) with standard errors are shown. Responses are compared to non-immunized control mice (Control).*, p<0.05 vs. non-immunized; **, p<0.05 vs. BCG alone; ***, p<0.05 CFA vs. Lactoferrin group.

FIG. 5 shows that BCG vaccination with adjunct Lactoferrin adjuvant limits mycobacterial dissemination following aerosol challenge with MTB. Mice immunized one time with BCG in IFA (BCG/IFA), BCG in IFA with Lactoferrin (100 μg/mouse) (BCG/IFA/LF), or BCG in CFA (BCG/CFA) were aerosol challenged with approximately 100 CFU virulent MTB, strain Erdman. Non-immunized controls are indicated (Control). 28 days post infection lung tissue (left) was assessed for bacterial load and spleen tissue (right) was examined for dissemination of organisms to peripheral tissue. Individual CFU values for 4 mice per group are shown; bars indicate average for group. Responses are compared between groups. *, p<0.05 vs. non-immunized. 3 of 4 mice in the Lactoferrin group exhibited sterilizing immunity in the spleen; values were placed at organism limit of detection (Log10 of 500 CFU/organ=2.69). Data representative of two experiments with similar results and reduction of CFU.

FIG. 6 Bacterial load post-challenge with MTB in mice immunized with BCG and lactoferrin adjuvant: C57BL/6 were immunized once with BCG, or with BCG and lactoferrin, formulated in saline, and subsequently aerosol challenged with Erdman MTB 4 weeks later. Mice were sacrificed at days 7, 28, and 65 post-challenge, and lung (top, A) and spleen (bottom, B) tissue were assessed for organ bacterial load. Comparisons were also made to non-immunized control infected mice. At least 6 mice were infected for each group. Average CFU per organ ± standard deviation shown; *p<0.05, **p<0.01, ***p<0.001 compared to non-immunized control infected mice at indicated times.

FIG. 7 shows that BCG admixed with Lactoferrin increased antigen specific IFN-γ production during recall response. Splenocytes were isolated from mice immunized with 10⁶ BCG/mouse with or without 100 μg/mL of Lactoferrin (boosted at 2 weeks) at 6 weeks post-boost and stimulated with Heat Killed-BCG (10:1). Supernatants were collected at 72 hours and analyzed by ELISA.

FIG. 8 shows that BCG vaccination with adjunct Lactoferrin adjuvant limits MTB induced pathological damage. Mice were immunized twice with BCG, or with BCG and Lactoferrin. 4 weeks after final immunization, mice were aerosol challenged with approximately 100 CFU virulent Erdman MTB. Mice were sacrificed 65 days post challenge, lungs were isolated and sectioned for histology (H&E, 40× and 100×). Control (non-immunized) mice also shown.

FIG. 9 Quantitative analysis of inflammation in immunized mice. Lung weight index (LWI) was calculated at day 7, 28 and 65 post challenge of mice immunized with BCG or BCG/lactoferrin, and compared to non-immunized challenged mice (top, A). On day 65, histological sections were assessed for changes in lung pathology by quantitative evaluation of inflammatory lesions, represented as percent occlusion of tissue (bottom, B). Results are shown as average standard deviation for at least 6 mice per group; *p<0.05, **p<0.01, compared to non-immunized control mice.

### BRIEF DESCRIPTION OF THE INVENTION

As presented in parent application, the invention provides novel techniques which can be employed for enhancing effectiveness of vaccination protocols. More specifically, the present invention is directed to the use of lactoferrin as an adjuvant to enhance effectiveness of vaccine, and/or reduce disease related pathology manifested after subsequent infection with infectious organisms. In addition, the present invention and vaccination regimens can be used to augment immune responses to directly treat (therapeutic) or prevent (prophylactic) manifestations of cancer, autoimmune disorders, and allergic response.

Lactoferrin, a mammalian glycoprotein, is generally considered an important nonspecific host defense component for protection against various pathogens. It bridges innate and adaptive immune function by regulating target cell response. The importance for lactoferrin as a bridge between innate and adaptive immune function can clearly be seen during elicitation of response during vaccination. Lactoferrin indeed is capable of enhancing the T-cell mediated delayed type hypersensitivity (DTH) response, as measured by foot pad swelling, to a variety of antigens, including sheep red blood cells (SRBC), ovalbumin (OVA), and Mycobacterium bovis Calmette-Guerin (BCG) (Zimecki M. Kruzel M. 2000. Systemic or local co-administration of lactoferrin with sensitizing dose of antigen enhances delayed type hypersensitivity in mice. Immunology Letters, 74:183-188.; Actor J., Hwang S. A., Olsen M., Zimecki M., Hunter R., Kruzel M. 2001. Lactoferrin immunomodulation of DTH response in mice. Int Immunopharmacol. 2002 March; 2(4):475-86.).

Human milk is high in lactoferrin content. In fact, the high degree of iron absorption from human milk is manifested in a low incidence of iron deficiency anemia among breast fed infants compared to infants fed with cow's milk. Accordingly, lactoferrin is a key protein for healthy development of infants. The severely limited amount of human milk however, restricts the large scale lactoferrin production. Furthermore, production of lactoferrin from human milk presents a risk factor of infectious contamination. That is, it could carry with it a potentially lethal contaminant, such as the human immunodeficiency virus (HIV) or another undesirable agent.

Accordingly, the present invention provides for the use of human recombinant lactoferrin, which is free of naturally occurring contaminants, e.g., proteins and viruses, that would be detrimental to the recipient, in particular, when given systemically (e.g. parenterally). Of particular interest for systemic human applications would be a human lactoferrin produced in an expression system which provides the glycosylation of human type. The technology for the “humanized” glycosylation has recently been reviewed in Nature/Microbiology (Stefan Wildt and Tillman Gengross. The humanization of N-glycosylation pathways in yeast. Nature Review 2005, 3:119-127) and in PNAS (Choi B K, Bobrowicz P, Davidson R C, Hamilton S R, Kung D H, Li H, Miele R G, Nett J H, Wildt S, Gerngross T U. Use of combinatorial genetic libraries to humanize N-linked glycosylation in the yeast Pichia pastoris. Proc Natl Acad Sci USA. 2003; 100:5022-5027).

The preferred recombinant lactoferrin is lactoferrin expressed in a yeast expression system such as Pichia pastoris or Hansenula polymorpha, or in a eukaryotic expression system., and is fully “humanized” with both amino acid sequence and sugar composition typical for human lactoferrin. The preferred lactoferrin is described in U.S. Pat. No. 6,066,469, entitled “Cloning, Expression and Uses of Human Lactoferrin” and its two divisional applications U.S. Pat. No. 6,277,817 B1 and 6,455,687 B1, both entitled “Human lactoferrin”.

Although lactoferrin contemplated for use in accordance with the present invention is preferably of human origin, more preferably via DNA recombinant means, most preferably fully “humanized” recombinant lactoferrin, but other lactoferrins, such as natural or recombinant bovine, goat and porcine lactoferrins, isolated and purified using methods readily available for the skilled in art, are contemplated. Lactoferrin administration before, during, or after vaccination is contemplated based on the specific antigen used for the vaccine. The preferred administration is when lactoferrin is delivered with a vaccine simultaneously.

According to the present invention lactoferrin is used to augment vaccine efficacy and subsequent protection against challenge of virulent pathogens. These may include lactoferrin adjuvant augmentation of vaccines against: Anthrax, Bacille Calmette-Guérin (BCG), Chickenpox, Diphtheria, Hepatitis A, Hepatitis B, Haemophilus influenzae type b (Hib), Human papillomavirus-HPV, Influenza, Measles, Meningococcus, Mumps, Pertussis, Pneumococcal/adults, Pneumococcal/children, Polio, Rabies, Rotavirus, Rubella, Smallpox, Tetanus, Tuberculosis, Varicella Zoster. Lactoferrin can also be used in vaccines to develop protective antibody responses to neutralize bacterial toxins, such as tetanus, and other bacterial produced toxins.

Also, lactoferrin is consider for use to augment vaccine efficacy and subsequent protection against challenge of the following parasitic species: Spriochets (Treponema, Borrelia), Obligate Intracellular Pathogens (Chlamydia, Rickettsia, Ehrlichia and Coxiella), Intestinal Protozoa (Amebae, Ciliates, Flagellates, and Coccidia), Extraintestinal Protozoa (Plasmodium, Babesia, Trypanosomes, Leishmania, Trichomonas, Acanthamoeba and Naegleria), Nematodes (Pinworms and Whipworms, Ascaris, Hookworm, Strongyloides, Trichinella, and Filariae), Cestrodes and Trematodes (Tapeworms and Schistosomes).

According to the present invention lactoferrin is used to augment the efficacy of cancer prophylactic and/or therapeutic vaccines and subsequent protection against such cancer or cancers. In particular, the two FDA approved cancer prophylactic vaccines against hepatitis B virus (associated with liver cancer) and human papilliomavirus (associated with cervical cancer) are contemplated.

Lactoferrin can also be used to augment the efficacy of vaccine against allergens. In particular, it is effective against, but not limited to, vaccines designed to limit allergic response to the ragweed pollen antigen.

According to the present invention lactoferrin is used to augment tolerogenic responses for the autoimmune disorders, using antigen-specific immature dendritic cells. This may be used in both prevention and therapeutic methods to limit presentation and manifestation of such disorders.

In still another embodiment of the present invention there is provided lactoferrin's ability to elicit in vitro and in vivo responses to BCG.

A more complete understanding can be obtained by reference to the following specific Examples. These Examples are described solely for the purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

Methods of immunology and biochemistry used but not explicitly described in this disclosure and these Examples are amply reported in the scientific literature and are well within the ability of those skilled in the art.

Cell lines: Two murine macrophage cell lines, J774A.1 and RAW 264.7, purchased from American Type Culture Collection (ATTC), were used for stimulation experiments. Cells were maintained in Dulbecco's Modified Eagle's Medium (DMEM, Sigma, St. Louis, Mo.) supplemented with 10% heat-inactivated fetal bovine serum (FBS, Sigma) and 0.01% HEPES (Sigma) and L-Arginine (Sigma). Macrophages were plated into 24 well plates at 1×10⁶ cells/mL/well using the media as outlined in 3.1. Triplicate cells of macrophages were stimulated with 200 ng/mL of lipopolysaccharide (E. coli 0111:B4 LPS; approximately 3×10⁶ E.U./mg) (Sigma), with increasing concentrations of bovine Lactoferrin (1 □g, 10 □g, 100□g, 500 □g, and 1 mg/mL), with Lactoferrin and LPS. Low endotoxin bovine Lactoferrin utilized was characterized as <1 E.U./mg, less than 25% iron saturated, and >95% purity. Cells were incubated at 37° C. with 5% CO₂. Supernatants were collected after 72 hrs and frozen at −20o C. until analyzed by ELISA for cytokine production.

Microorganisms: The BCG Pasteur strain (TMC 1011, ATCC, Manassas, Va.) was grown in Dubos base (without addition of glycerol) with 10% supplement (5% BSA and 7.5% dextrose in saline) on an orbital shaker at 37° C. for two weeks before use. The Erdman strain of Mycobacterium tuberculosis (TMC 107, ATCC) used for challenge was grown in Dubos base (with 5.6% glycerol) with 10% supplement for three weeks before use. Cultures were taken during log growth period. Organisms were washed with 1×PBS (Dulbecco's Phosphate buffered saline 10×, Cellgro, Herndon, Va.) and resuspended in 1×PBS. Suspensions were sonicated for 5 seconds prior to use. Bacterial concentration was determined using McFarland standards, and confirmed by plating dilutions onto 7H11 agar plates (Remel, Lenesa, Kans.). Plates were incubated at 37° C. with 5% CO2 for 3 to 4 weeks, and colonies were enumerated. Heat-killed BCG (HK-BCG) was produced by autoclaving the BCG suspension in 1×PBS at 121° C. for 20 min. Death of BCG was verified by plating of autoclaved BCG on 7H11 plates.

Vaccination: C57BL/6 female mice (8-10 weeks) were immunized with 100 μL of vaccine formulation (10-20 mice/group), subcutaneously, at the base of the tail. BCG was given at 1×10⁶-1×10⁷ CFU/mouse, and lactoferrin was given at 100 μg/mouse. For the emulsion formulation, BCG with or without lactoferrin was emulsified with incomplete Freund's adjuvant (IFA) (Difco, Detroit, Mich.) in a 1:1 (v/v) ratio. For the saline formulation, BCG with or without lactoferrin was combined in 1×PBS (“saline”). Mice were vaccinated a single time; a group of mice were boosted at indicated times post initial immunization. Experiments were repeated at least two times, using 6 or more mice/group per sacrifice time point.

Antigenic Responses: 17 days post-immunization, spleens were harvested from each immunization group, homongenized, and red-blood cells lysed by ACK buffer (Cambrex Bio Science, East Rutherford, N.J.). Splenocytes were washed 2× with 1×PBS and resuspended in DMEM, supplemented with 10% heat-inactivated FBS, 0.005% 2-mercaptoethanol (2Me, Gibco, Carlsbad, Calif.), 0.01% penicillin G (Sigma) and Gentamycin (Sigma), 0.01% HEPES (Sigma) and L-Arginine (Sigma). Cells were plated at 10⁶ cells/mL and stimulated with HK-BCG at 5:1 ratio. 4 to 6 mice were used per group. Assay was done in duplicate or triplicate. Supernatants were collected at 24 and 72 hrs and frozen at −20° C. until evaluation by ELISA. Recall response experiments were repeated a total of 3 times with similar results.

Proliferation Assay: Splenocytes at 5×10⁵ cells/100 μL/well were plated in DMEM without phenol red (Sigma) and stimulated with HK-BCG at 5:1 ratio. After 48 hours, proliferation was measured using the MTT assay (Sigma) following manufacture protocols. MTT was dissolved in 1×PBS at 5 mg/mL and filtered through a 0.2 μm filter. 10 μL of MTT solution was added to each well, and plates were incubated at 37o C. for 4 hours. Supernatants were removed and 100 μL of 0.1N of hydrochloric acid (HCl) in anhydrous isopropanol was added. 4 to 6 mice were used per group; assay was performed in triplicate. Absorbance was read at 570 nm subtracting background at 690 nm. Proliferation Index was calculated relative to naïve control splenocytes in the absence of HK-BCG stimulation.

ELISA: Supernatants were assayed in triplicate for cytokine production using enzyme-linked immunosorbent assay (DuoSet ELISA kit, R&D Systems, Minneapolis, Minn.), according to manufacture's instructions. Supernatants were analyzed for T-cell cytokine IFN-γ, cellular mediators of TH1 response (IL-12p40, IL-10), and proinflammatory mediators (IL-1β, IL-6, TNF-α). IL-1β and TNF-α were analyzed at 24 hrs, all others at 72 hrs. Values (pg/ml) were by regression analysis of data to standard curves generated.

Erdman challenge: 14 days post-immunization, 4 mice from each group were aerosol challenged with Erdman MTB (13). Each mouse was infected via aerosol with <100CFU/mouse. Aerosol infection was achieved using an inhalation exposure system (IES) (GLAS-COL Model #A4212 099c Serial # 377782). Verification of infectious dose was accomplished at 1 day post-infection; 4 mice were sacrificed, lungs collected and homogenized, and dilutions plated onto 7H11 agar plates for CFU counts. 28 days after Erdman challenge, mice were sacrificed. Lung and spleen tissues were isolated, homogenized, and plated on 7H11 agar plates for CFU determination. Plates were incubated at 370C for 3 to 4 weeks before enumeration.

Histological techniques: The histological analysis of the lung was performed on day 28 and day 65 following aerosol infection. Lungs were fixed in 10% formalin and embedded in paraffin using standard techniques. Sections, 5 μm thick, were stained with hematoxilin and eosin (H&E) and subsequently reviewed histologically; the pathologist viewing and interpreting the slides was blind to the type of experiment and treatment. Lungs from at least 6 mice of each group were analyzed. On day 65, histologic analysis of occlusion was performed in a blinded manner. 2-3 sections per block from 6-10 mice per time point for each group were analyzed using digital software (NIH Image, Prism-GraphPad).

Statistics: One way ANOVA comparison or Student's t-test were used for data analysis. Statistical significance was assigned for values of p<0.05 or as indicated, comparing average values and standard errors between groups.

These data demonstrate that lactoferrin given as an adjuvant composition with BCG vaccine is suitable to augment the efficacy of such vaccine as well as to reduce the pathology (organ injury) when the subject is challenged with a virulent pathogen.

### EXAMPLE 1

Lactoferrin Enhances IL-12 Production from Stimulated Macrophages.

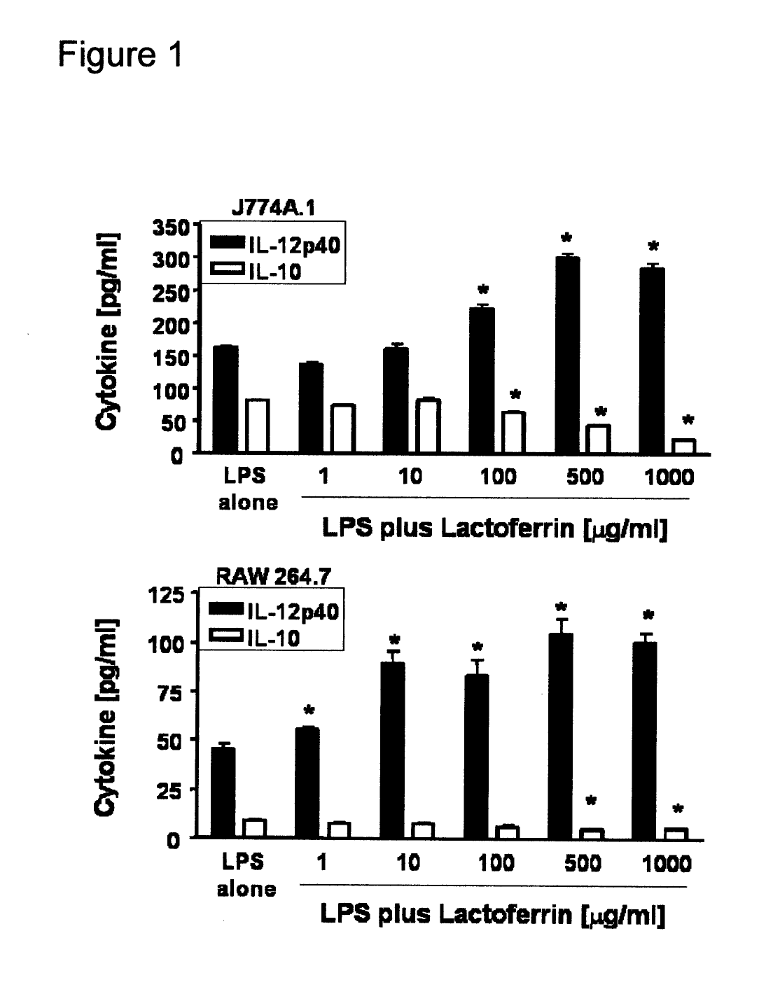

Lactoferrin was examined for the ability to enhance IL-12 production in vitro from LPS stimulated macrophage cell lines. J774A.1 or RAW 264.7 cultured macrophages were stimulated with LPS (200 ng/mL) in the presence of increasing Lactoferrin concentrations (FIG. 1). Stimulation of both cell lines with LPS led to production of IL-12(p40), with J774A.1 cells slightly more responsive than the RAW 264.7 line. Addition of Lactoferrin led to significant (p<0.05) increase in IL-12 from both cell lines. Increased IL-12 was apparent at 100 μg/ml of Lactoferrin and above. The RAW 264.7 cells were more sensitive to Lactoferrin treatment with increased IL-12 production at all Lactoferrin concentrations tested from 1 μg/ml through 1000 μg/ml. J774A.1 or RAW 264.7 macrophages did not produce any significant levels of IL-12p40 without stimulation with LPS or when stimulated with Lactoferrin alone (not shown).

In a converse relationship, Lactoferrin reduced the production of IL-10 from LPS stimulated macrophages (FIG. 1). The J774A.1 cell line produced approximately 81 μg/ml IL-10; addition of 100 μg/ml Lactoferrin and above led to significant (p<0.05) decrease. Although the levels of IL-10 produced upon LPS stimulation from the RAW 264.7 cell line was lower, Lactoferrin was able to reduce IL-10 production at higher concentrations. J774A.1 or RAW 264.7 macrophages did not produce any significant levels of IL-10 without stimulation with LPS or when stimulated with Lactoferrin alone (not shown).

Overall, Lactoferrin was able to affect LPS stimulation of macrophage cell lines, resulting in an increased ratio of IL-12:IL-10 production, leading to increased production of mediators necessary for driving TH1 mediated functions. High IL-12:IL-10 balance is critical in the development of T cell responses that aid in protection against tuberculosis, and against many other pathogens. Vaccination protocols that push responses towards high IL-12 with relatively lowered IL-10 are considered effective. Lactoferrin increase in this response augments vaccine efficacy.

### EXAMPLE 2

Lactoferrin Enhances In Vivo Development of TH1 Mediators to BCG Antigen—Generation of T Helper Cell Response.

Lactoferrin was examined for ability to effectively increase BCG immunization for promotion of TH1 immunity. Immunization conditions were stringent to investigate early events in generation of response. Mice were immunized only once s.c. with 10⁶ BCG emulsified in IFA, in IFA and Lactoferrin, or in CFA. Splenocytes were harvested 17 days post-immunization and stimulated in vitro with Heat-Killed-BCG (HK-BCG) at a ratio of 5:1. Supernatants were collected after 72 hrs and analyzed for IL-12(p40) and IL-10 (Table I).

TABLE I
BCG Immunization with adjunct Lactoferrin adjuvant increases IL-
12(p40):IL-10 ratios (with standard deviation).

| | IL-12p40 | IL-10 | Ratio |
|---|---|---|---|
| Group | [pg/ml] | [pg/ml] | IL12p40:IL10 |
| Non-immunized | 45 (4) | 61 (4) | 0.77 (0.09) |
| BCG/IFA | 50 (11) | 69 (9) | 0.70 (0.1) |
| BCG/IFA/ | 86 (11)*^{/}** | 73 (18) | 1.43 (0.22)*^{/}** |
| Lactoferrin | | | |
| BCG/CFA | 69 (1.5)*^{/}** | 93 (7)*^{/}** | 0.78 (0.07) |

A single administration of BCG in IFA and Lactoferrin resulted in significantly increased production of IL-12 in the splenic recall assay (p<0.05), with 86 pg/ml produced. The high IL-12:IL-10 balance would assist development of T cell responses that protect against tuberculosis. Vaccination protocols that push responses towards high IL-12 with relatively lowered IL-10 are considered effective. Lactoferrin increase in this response augments vaccine efficacy.

Likewise, the CFA positive control immunization group also demonstrated significant increase in IL-12 production, relative to non-treated mice. In comparison, with only one immunization and short time to recall, the group receiving BCG emulsified in IFA alone did not generate significantly increased IL-12 production (50 pg/ml). Evaluation of IL-10 was also performed; immunization with Lactoferrin did not significantly increase production of this cytokine. Splenic recall to HK-BCG demonstrated only modest increases in IL-10 production for both the IFA alone and IFA with Lactoferrin groups. In contrast, BCG administered in CFA demonstrated significant increase in IL-10.

A comparison of IL-12 to IL-10 produced revealed strong and significant shift in response generated between groups. Immunization in IFA and Lactoferrin adjuvant demonstrated a significantly higher ratio of IL-12:IL-10 for the IFA and Lactoferrin group (ratio of 1.43) compared to either IFA or CFA alone (ratios of 0.70 and 0.78 respectively).

### EXAMPLE 3

Lactoferrin Enhances In Vivo Development of Pro-Inflammatory Mediators and IFN-γ.

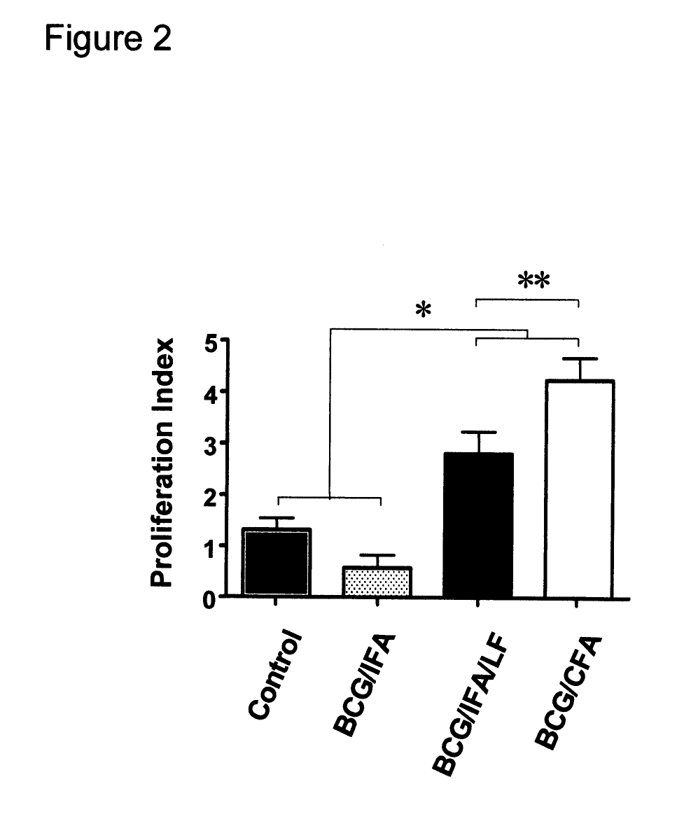

Splenocytes from Lactoferrin immunized mice demonstrated strong proliferative response to HK-BCG (FIG. 2). A comparison of stimulation index (SI) revealed specific proliferation to antigen was significantly enhanced for the IFA with Lactoferrin and for the CFA immunized groups (2.81±0.43 and 4.24±0.42) versus the non-immunized group (1.31±0.23) or BCG emulsified in IFA group (0.58±0.25).

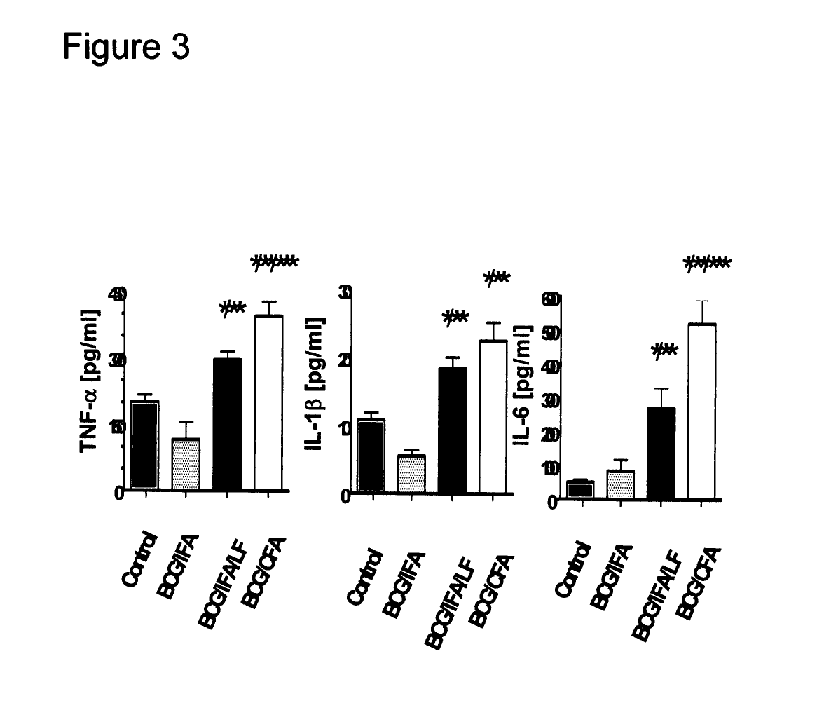

The assay was extended to further examine generation of proinflammatory response (TNF-α, IL-β and IL-6) in antigenic recall to HK-BCG. Splenocytes from individual mice were stimulated with HK-BCG (5:1). Supernatants were collected and assayed (FIG. 3). In concert with the increased stimulation index, there was significant production of all three proinflammatory mediators in the Lactoferrin immunization group compared to both the non-immunized and IFA immunized groups (p<0.05). Likewise, the CFA immunization group remained statistically higher for TNF-α, IL-β and IL-6, with TNF-α and IL-6 markedly elevated compared to all other groups.

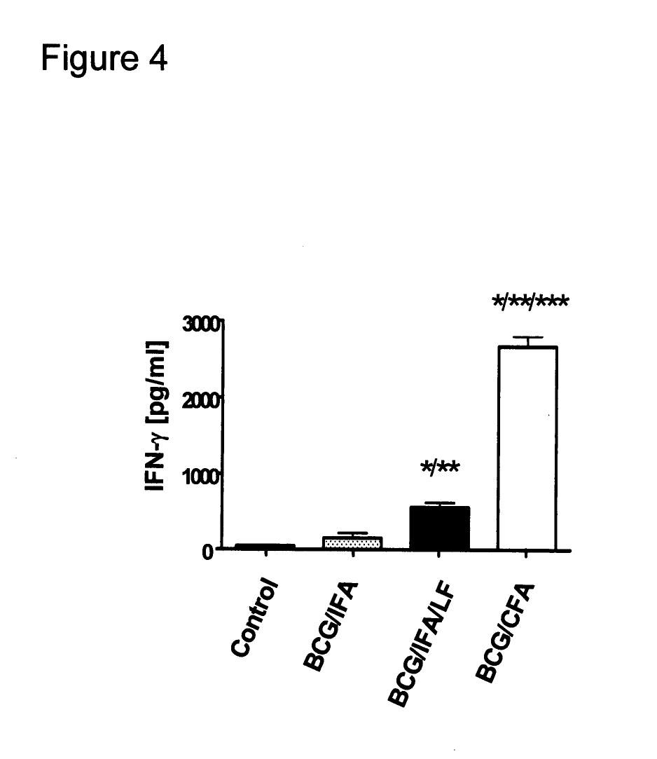

IFN-γ response was measured as a direct indicator of generated TH1 response, and as a marker for required function of vaccine efficacy to protect against virulent mycobacterial infection (FIG. 4). High levels of IFN-γ produced from antigen specific T cells is required for protection against tuberculosis. Vaccination with Lactoferrin adjuvant increased IFN-γ production from T cells from immunized animals. Increased IFN-γ was found upon in vitro stimulation with HK-BCG. Specifically, the IFA/Lactoferrin group produced 556.2±63.8 pg/ml IFN-γ compared withl49.5±7.6 pg/ml for the IFA alone group. As expected, the positive control CFA adjuvant group generated the highest level of IFN-γ with 2668.0±136.6 pg/ml.

### EXAMPLE 4

Vaccination with Lactoferrin Adjuvant Increases Protection Against Challenge with Virulent M. tuberculosis.

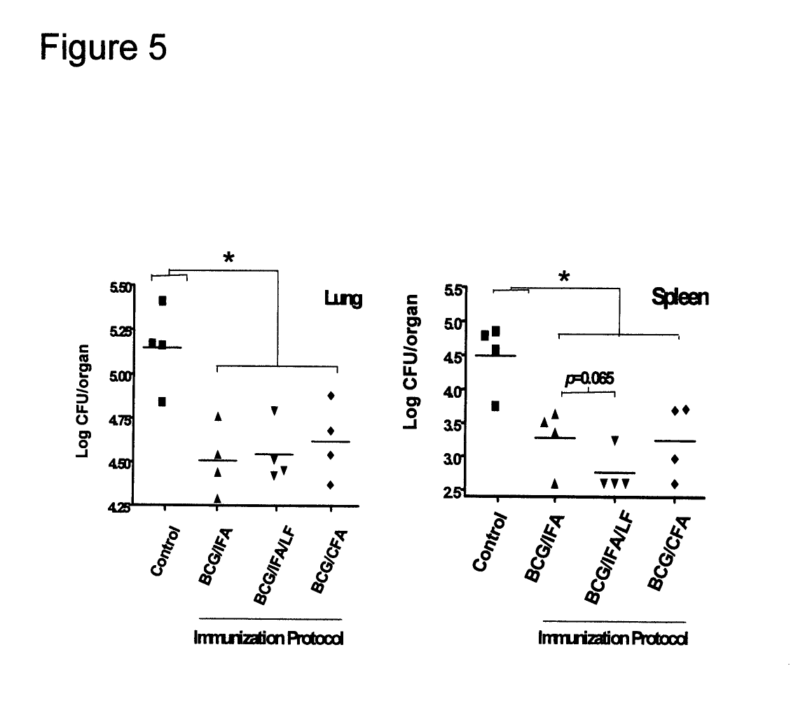

Immunized mice were aerosol challenged with virulent Mycobacterium tuberculosis, strain Erdman (<100 CFU per lung). Tissue was obtained four weeks following implantation and CFU were enumerated. All immunization groups showed significant reduction in lung organism load (p<0.05) compared to the non-immunized control, with nearly 1 log less organisms present indicating local growth control within the tissue of implantation (FIG. 5, left). Of major importance is the ability to restrict organism dissemination to peripheral organs following aerosol challenge. In this regard, CFUs enumerated from spleen tissue detailed differences between immunization groups (FIG. 5, right). In the spleen, the non-immunized mice showed 4.49±0.25 log CFU, indicating spread of organisms to that tissue. In contrast, all BCG immunized groups limited dissemination. The IFA and Lactoferrin adjuvant group demonstrated the largest and most consistent reduction (p<0.065) in bacterial load within the spleen (2.760±0.32 log CFU); three of 4 mice demonstrated sterilizing immunity in that tissue with CFU levels below the limit of detection (500 CFU per organ). Indeed, the Lactoferrin immunized group further reduced bacterial loads compared to the IFA alone group (3.278±0.23 log CFU) and the CFA group (3.243±0.27 log CFU). At this day 28 time point, no detectable organisms were found in the liver for any group, including the non-vaccinated controls (data not shown).

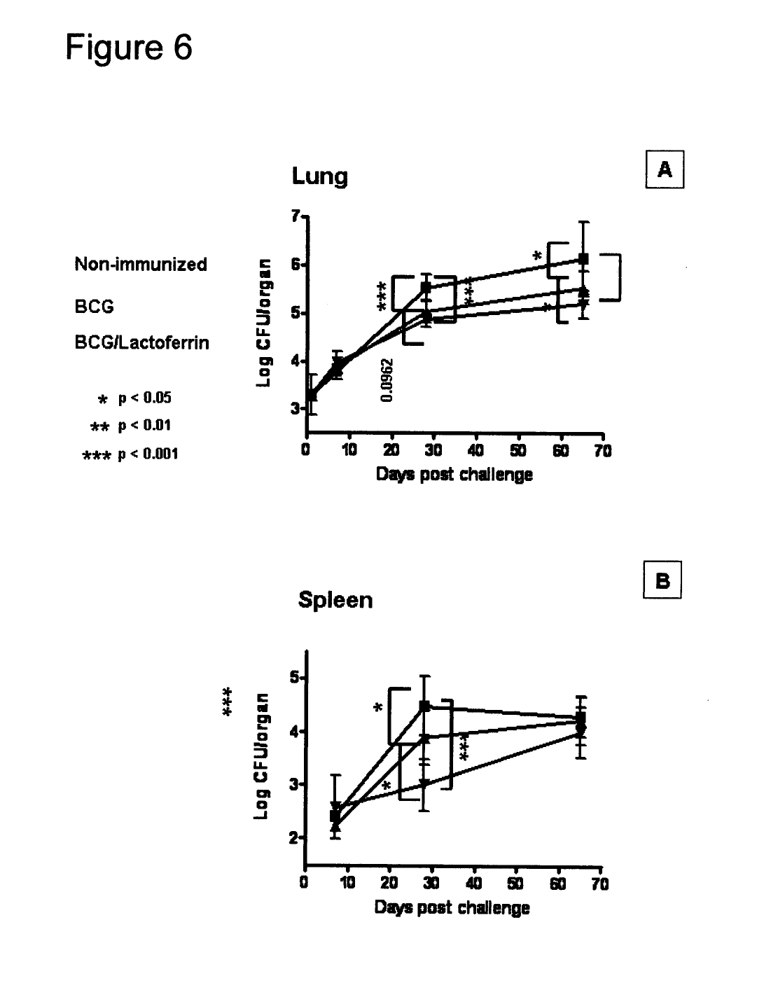

A longer term study showed similar results, with long term protective effects in mice immunized with BCG and lactoferrin, relative to non-immunized or BCG alone immunized mice. (FIG. 6). At day 28 post-challenge, a significant (p<0.001) decrease in lung CFU (FIG. 6, top) was observed in mice immunized and boosted with BCG (5.03+/−0.22 Log CFU/organ) and BCG/lactoferrin (4.88+/−0.16 Log CFU/organ) when compared with the non-immunized (5.54+/−0.27 Log CFU/organ) group. At day 65 post-challenge, mice immunized and boosted with BCG (5.54+/−0.34 Log CFU/organ) or BCG/lactoferrin (5.19+/−0.29 Log CFU/organ) significantly (p<0.05) decreased lung bacterial load compared to the non-immunized group (6.13+/−0.77 Log CFU/organ). In addition, the decrease in lung CFU from mice immunized and boosted with BCG/lactoferrin was significant (p<0.05) compared to the BCG immunized and boosted group. Examination of splenic CFU (FIG. 6, bottom) at day 28 post-challenge showed mice immunized and boosted with BCG (3.9+/−0.51 Log CFU/organ) or with BCG/lactoferrin (3.00+/−0.49 Log CFU/organ) decreased splenic CFU compared to the non-immunized group (4.49+/−0.55 Log CFU/organ). Mice immunized and boosted with BCG/lactoferrin had a further 1 Log decrease in splenic CFU compared to mice immunized and boosted with BCG only.

### EXAMPLE 5

Lactoferrin Adjuvant Admixed with BCG Vaccine Increases Antigen Specific IFN-γ Production and Limits Tuberculosis Related Pathology (Reduction of Organ Injury).

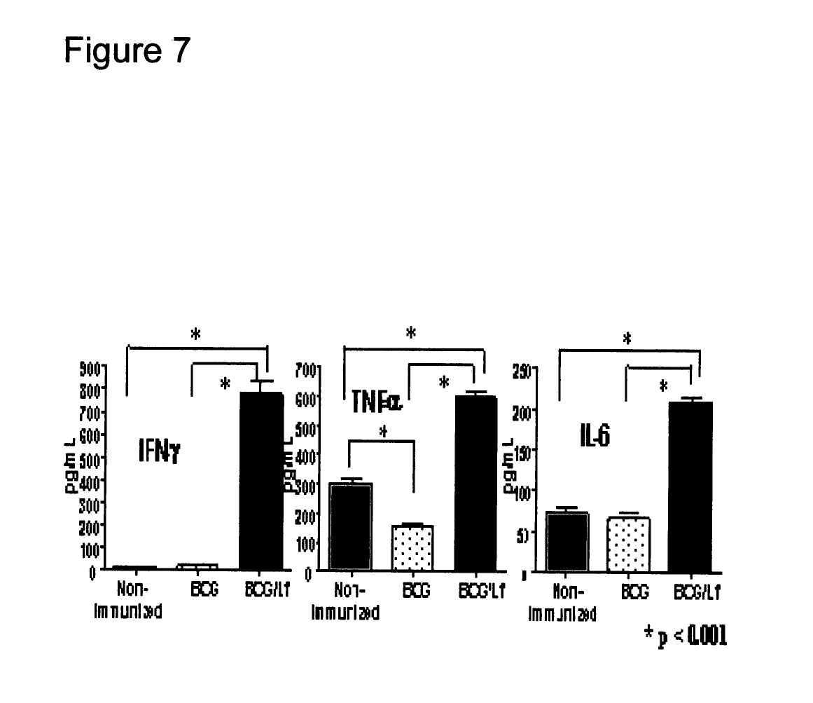

Mice were immunized subcutaneously (s.c.) at the base of the tail and boosted at 2 weeks. The immunization groups were: 1) non-immunized, 2) 10⁶ BCG (ATCC), and 3) 100 ug of Lactoferrin with 10⁶ BCG. FIG. 7 shows that BCG admixed with Lactoferrin increased antigen specific IFN-γ production during recall response. 10⁶ Splenocytes were isolated from mice immunized with 10⁶ BCG/mouse with or without 100 μg/mL of Lactoferrin (boosted at 2 weeks) at 6 weeks post-boost and stimulated with Heat Killed-BCG (10:1). Comparisons were made to non-immunized controls. Supernatants were collected at 72 hours and analyzed by ELISA for IFN-γ, TNF-α and IL-6. For all three cytokines tested, the immunization with BCG and lactoferrin generated significant recall responses from splenocytes which was significantly greater in magnitude to responses generated by splenocytes isolated from BCG alone immunized mice, or from splenocytes isolated from non-immunized controls. This confirms the ability to generate specific IFN-γ producing cells in mice immunized in the presence of lactoferrin adjuvant.

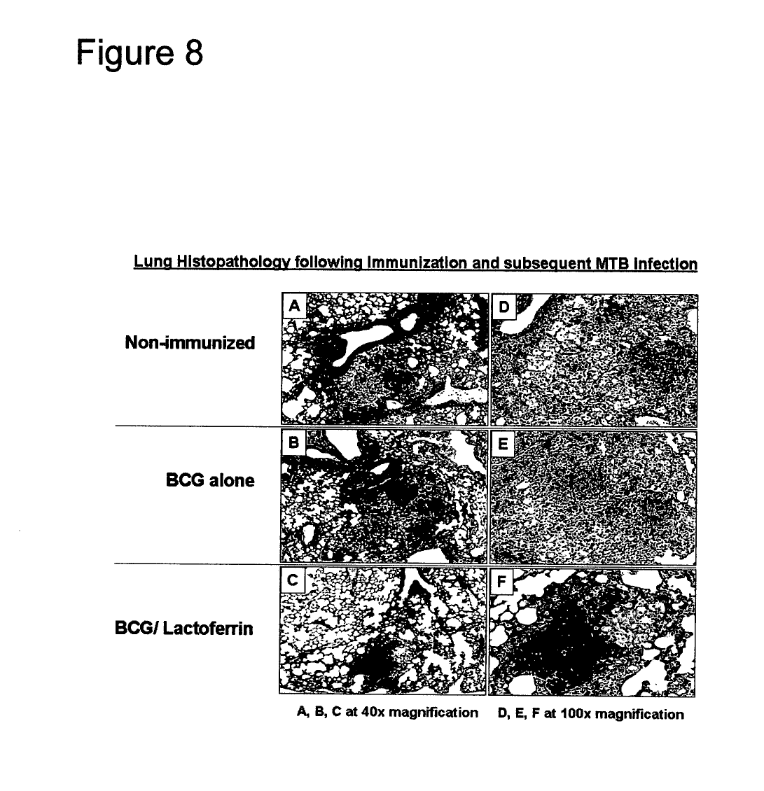

Lactoferrin adjuvant also increased the protection against subsequent challenge with virulent organisms. Mice were immunized subcutaneously (s.c.) at the base of the tail and boosted at 2 weeks. There were 20 mice per immunization group: 1) non-immunized, 2) 10⁶ BCG (ATCC), and 3) 100 ug of Lactoferrin with 10⁶ BCG. Four weeks after boosting, mice were infected with approximately 100 CFU aerosolized Erdman Mycobacterium tuberculosis (MTB) (ATCC). Each mouse was aerosol infected with approximately 100 organisms. Mice were sacrificed at day 7, 28 and 65 post-challenge. Lung and spleen were isolated and plated on 7H11 plates (Remel) for colony forming units (CFU) assessments. Sections were taken for histological study (H&E staining). Lungs from non-immunized control mice (FIG. 8A, 8D) and lungs from mice immunized with BCG alone (FIG. 8B, 8E) demonstrate granulomatous responses with marked destructive pathology. The group immunized with BCG and lactoferrin (FIG. 8C, 8F) demonstrated reduced manifestation of pathological organ injury and controlled responses to infection within lung tissue.

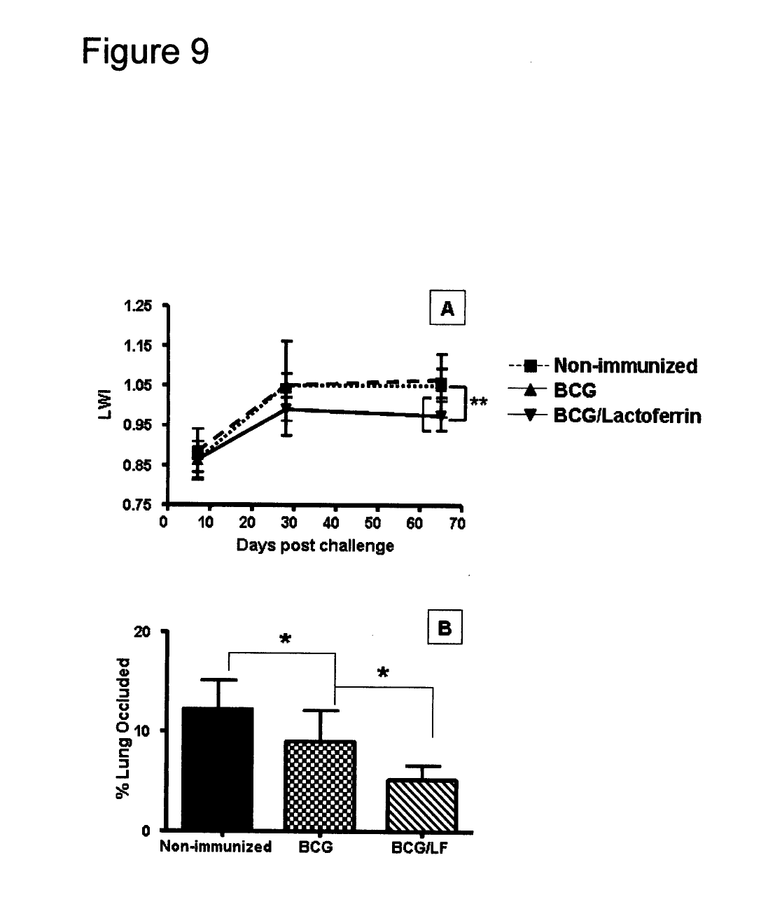

Immunization with BCG and lactoferrin demonstrated better histopathology non-immunized or BCG alone immunized mice. The apparent reduction in inflammatory pathology was semi-quantitated by analysis of lung weight index (LWI), and quantitatively by measuring the area of the lung occluded by granulomas and lesions developed during the infective process. Mice immunized and boosted with BCG in the presence of lactoferrin had consistently lower LWI, at days 28 and 65 compared to mice immunized with BCG and the non-immunized group (FIG. 9, top). An analysis was undertaken to quantitate changes in pathological manifestation due to infection. The BCG/lactoferrin immunized mice demonstrated significant reduction in lung occlusion at 65 days post infection with the virulent organisms (FIG. 9 bottom).

The specific lactoferrins used in the present invention are illustrated in the following Examples:

### EXAMPLE 6

Bovine milk lactoferrin (BLF): Bovine milk lactoferrin is a highly purified Lactoferrin derived from cow's milk. The commercially available BLF (Glanbia Nutritionals, Twin Falls, Id., USA) is further purified by metal ion affinity chromatography (IMAC). An imminodiacetic acid-epoxy activated gel (Pharmacia Fine Chemicals, Uppsala, Sweden, CHELATING SEPHAROSE™ 6B) is washed with water and equilibrated with 0.1 M sodium acetate buffer (pH 4.0) containing 1 M sodium chloride. The gel is then packed into a chromatographic column (1.2 cm×10 cm) and saturated with 4 bed volumes of the same sodium acetate buffer further containing 5 mg/ml of nickel chloride. Excess metal is washed from the column with the sodium acetate buffer, and the gel is equilibrated with 20 mM HEPES buffer (pH 7.0) containing 1 M sodium chloride and 2 mM imidazole. The commercially available BLF is mixed with HEPES, sodium chloride, and imidazole to obtain a pH of 7.0, 20 mM HEPES, 1 M sodium chloride, and 2 mM imidazole. The mixture is applied onto the column at a flow rate of about 1 ml/min followed by, washing the gel with 2 bed volumes of 20 mM HEPES buffer (pH 7.0) containing 1 M sodium chloride and 2 mM imidazole. The non-adsorbed fraction is discarded, and the adsorbed fraction containing lactoferrin is eluted using 2 bed volumes of 20 mM HEPES buffer (pH 7.0) containing 1 M sodium chloride and 20 mM imidazole. The final material is dialyzed against water and lyophilized. BLF obtained in this example is at least 95% pure and is free of Coliform bacteria, Salmonella and pathogenic Staphylococcus. For oral administration BLF is reconstituted with water at concentration 0.5% (w/v) and stored at 4° C.

### EXAMPLE 7

Human milk lactoferrin (HML): Human milk lactoferrin is a highly purified lactoferrin derived from human milk. The commercially available lactoferrin (Sigma Chemical Company; St Louis, Mo.; USA) is further purified by metal ion affinity chromatography (IMAC) as described in Example 6. Alternatively, the gel is then packed into a chromatographic column (1.2 cm×10 cm) and saturated with 4 bed volumes of the same sodium acetate buffer further containing 5 mg/ml of copper sulfate. Excess metal is washed from the column with the sodium acetate buffer, and the gel is equilibrated with 50 mM TRIS HCL buffer (pH 8.0) containing 1 M sodium chloride. Lactoferrin is equilibrated to 50 mM TRIS HCL pH 8.0, 1 M NaCl and applied onto the column at a flow rate of about 1 ml/min followed by washing the gel with 2 bed volumes of 50 mM TRIS HCL buffer (pH 7.0) containing 1 M sodium chloride. The non-adsorbed fraction is discarded, and the adsorbed fraction containing lactoferrin is eluted using 2 bed volumes of 200 mM sodium acetate buffer pH 3.0. The eluate is neutralized with sodium hydroxide (final pH 7.2), sterilized by filtration and lyophilized. The final material is at least 95% pure. For the systemic administration the HML is reconstituted with sterile saline to obtain the final concentration of 10 mg/ml and is stored at 4° C.

According to the present invention lactoferrin used as an adjuvant may be human lactoferrin, bovine lactoferrin or recombinant human lactoferrin alone or in combination from the stock solutions described in example 6 and 7.

According to the present invention lactoferrin is administered either enteraly, preferably orally, in the form of a powder, aqueous or non-aqueous solution or gel, or parenterally, preferably intramuscularlly, in the form of an injectable solution, as an adjuvant to augment a vaccine efficacy and subsequent protection against challenge with a virulent pathogen. Preferable formulations of the present invention comprise lactoferrin alone or in combination with pharmaceutical or nutritional carriers such as, water, saline, starch, maltodextrin, pullulan, silica, talcum, stearic acid, its magnesium or calcium salt, polyethyleneglycol, arabic, xanthan or locoust bean gums and fatty emulsions and suspensions that will be readily apparent to the skilled artisan. The lactoferrin is preferably present in the formulation at a level of 0.01 milligram to 10 milligram, more preferably between 0.1 to 5 milligram, based on 1 milliliter or 1 gram of the carrier. An effective amount of lactoferrin varies depending on the individual treated and the form of administration. Preferable in treating individual, a single dose of 0.01 milligram to 20 milligrams, more preferable 0.1 milligram to 1 milligram of lactoferrin per kilogram of body weight is administrated. Lactoferrin can also be delivered as a liposomal formulation, including transdermal patches.

According to the present invention, lactoferrin can be incorporated as an adjuvant composition in formulation with any vaccine agent or adjunct therapeutic such as a) vaccines containing killed microorganisms—examples are vaccines against flu, cholera, bubonic plague, and hepatitis A; b) vaccines containing live, attenuated microorganisms—examples include yellow fever, measles, rubella, Bacille Calmette-Guérin (BCG) and mumps; c) toxoids—examples of toxoid-based vaccines include tetanus and diphtheria; d) subunit—examples include the subunit vaccine against human papillomavirus (HPV) that is composed of the viral major capsid protein; e) prophylactic or therapeutic cancer—examples include antigen BCG for prostate cancer treatment, or HPV antigens for cervical cancer; f) allergen vaccines—example include ragweed pollen vaccine; g) autoimmune vaccine—example include use of antigens to induce tolerance against self antigens, for example autoimmune responses leading to induction of myasthenia gravis Or other autoimmune reactions. Lactoferrin delivery can occur alone or simultanously per os, intravenously, intraperitonealy, intraarterialy, intramascularly, subcutanoeusly, transdermally, or as an intranasal spray, or intrabroncheal inhalation mist, at the effective concentration ranges set forth herein above.

Lactoferrin can be delivered before, after or concomitantly with vaccine or any other therapeutic agents providing however that the time between administration of the compositions (vaccine and adjuvant) is less than 24 hours, more preferably less than 12 hours. Preferred formulations of the present invention comprise incorporating the lactoferrin into a sterile aqueous solution as exemplified in Example 7.

It is understood that the examples and embodiments described herein are for illustrative purposes only and that various modifications or changes in light thereof will be suggested to persons skilled in the art and are to be included within the spirit and purview of this application and scope of the appended claims.

What is claimed:

1. A composition for augmenting vaccine efficacy in mammals comprising: a) lactoferrin as an adjuvant and b) antigen specific vaccine for use in a vaccination protocol.

2. A composition according to claim 1 wherein said lactoferrin is human.

3. A composition according to claim 1 wherein said lactoferrin is bovine.

4. A composition according to claim 1 wherein said antigen specific vaccine is tuberculosis (TB) vaccine.

5. A composition for protection against pathological manifestation in mammals and reduction of organ injury when challenged with an antigen, comprising: a) lactoferrin as an adjuvant and b) antigen specific vaccine, for use in vaccination protocol.

6. A method for augmenting vaccine efficacy in mammals comprising the steps of: a) administering an effective amount of pharmaceutically acceptable lactoferrin; and b) administering an effective immunizing amount of said vaccine.

7. The method according to claim 6 wherein said lactoferrin is given parenterally.

8. The method according to claim 6 wherein said lactoferrin is given enterally.

9. The method according to claim 6 wherein said lactoferrin is given prior to vaccine administration.

10. The method according to claim 6 wherein said lactoferrin is given after vaccine administration.

11. The method according to claim 6 wherein said lactoferrin is given simultaneously with the vaccine.

12. The method according to claim 6 wherein said lactoferrin is bovine.

13. The method according to claim 6 wherein said lactoferrin is human.

14. A composition comprising: a) human lactoferrin as an adjuvant to a vaccine for humans; b) and a vaccine used to vaccinate humans.

15. A composition according to claim 14 wherein said human lactoferrin is recombinant lactoferrin.
